(19) United States
(12) Patent Application Publication
(10) Pub. No.: US 2003/0004494 A1
(43) Pub. Date: Jan. 2, 2003

(54) Aspirator

(76) Inventor: Ralph Caselnova, Mont Claire, NJ (US)

Correspondence Address: Ralph Caselnova, 14 Linden Avenue, Mont Claire, NJ, 07042, US

(21) Appl. No.: 09/880,643

(22) Filed: Jun. 14, 2001

(51) Int. Cl. A61M 1/00

(52) U.S. Cl. 604/540; 604/514; 604/35; 604/66

(57) ABSTRACT

An aspirator (10) having a first tube (12) insertable in a patient's nose (18) through a patient's nasopharynx (20) and esophagus lumen (22) into a patient's stomach (24). The first tube (12) has a first tube first end (12A) and a first tube second end (12B). A second tube (14) insertable in the patient's nose (18) through the patient's nasopharynx (20). An activator (16) which has an activator sensor (16A) positioned within the second tube (14) at a distal end thereof.

### DESCRIPTION OF THE PRIOR ART

Aspirators are well known in the art. They come in various configurations, sizes and have numerous options. However, there are no aspirators with a sensor to detect fluid level within a patient's esophagus lumen.

Numerous innovations for an aspirator have been provided in the prior art that are described as follows. Even though these innovations may be suitable for the specific individual purposes to which they address, they differ from the present invention as hereinafter contrasted.

In U.S. Pat. No., 5,989,208, titled, Therapeutic ultrasound system, invented by Nita, an ultrasound system is disclosed having a catheter including an elongate flexible catheter body having at least one lumen extending longitudinally there through. The catheter further includes an ultrasound transmission member extending longitudinally through the lumen of the catheter body, the ultrasound transmission member having a proximal end connectable to an ultrasound transducer. The catheter also includes a distal head positioned on the distal end of the ultrasound transmission member. The catheter also has a coil with a proximal end connected to the distal end of the catheter body, and a distal end connected to the distal head. The coil defines a lumen communicating with the lumen of the catheter body. The ultrasound system also includes a sonic connector for coupling the ultrasound transmission member to the transducer. Different configurations are disclosed for the distal head and the ultrasound transmission member.

In U.S. Pat. No., 5,651,769, titled, Method for retrieving pancreatic juice utilizing and endoscopically wire-guided catheter, invented by Waxman, et. al., a method for accurately and quickly positioning a double lumen gastroduodenal tube into predetermined locations utilizing an endoscopically placed guide wire to retrieve duodenal fluid uncontaminated by gastric fluid, with little or no discomfort to the patient, and minimal radiation exposure. Specifically, an endoscope is passed through a patent's esophagus and stomach into the duodenum such that its distal end is located at a predetermined location, such as the fourth portion of the duodenum. While the endoscope is held in place, a guide-wire is passed through its center instrument channel to position a distal end of the guide-wire adjacent to the distal end of the endoscope. The endoscope is then withdrawn from the patient, leaving the guide-wire. A double lumen gastroduodenal tube is then threaded over the guide-wire such that gastric ports are aligned with the stomach and duodenal ports are aligned with the fourth portion of the duodenum so that upon aspiration of the gastric ports, the gastric fluid is prevented from entering the duodenum and contaminating the duodenal fluid.

In U.S. Pat. No., 5,456,251, titled, Remote sensing tonometric catheter apparatus and method, invented by Fiddian-Green, a tonometric device for use in hollow viscus tonometry and remote sensing of patient fluid parameters is disclosed in combination with a secondary apparatus, such as a urinary catheter, a nasogastric sump apparatus, or a feeding tube, for example. The device is capable of monitoring certain critical fluid properties of interest, such as oxygen gases and carbon dioxide gases in the wall tissue itself of the patient's organ, rather than monitoring such properties in a lumen of the organ. A walled sampling chamber, which is preferably an inflated balloon member, is provided on an elongated tube, with provisions for positioning the sampling chamber in direct contact with a wall portion of the patient's internal organ.

In U.S. Pat. No., 5,397,302, titled, Method of using a dual lumen biliary catheter, invented by Weaver, et. al., an improved biliary catheter is disclosed. The catheter comprises a tube constructed from a polyurethane or nylon having a durometer of about 60D and coated with a hydrophilic coating to provide lubricity, kink resistance and suppleness. The tube has substantially cylindrical sidewalls and a proximal end for connection to a source of contrast medium and a distal end for entry into the common biliary duct of a patient. The tube contains a first crescent-shaped lumen channel extending between the proximal and the distal end, the first lumen channel transporting the contrast medium from the source of contrast medium to the biliary duct; and a second circular lumen channel extending between the proximal end and said distal end for facilitating the insertion and threading of a spring wire guide into the dual-lumen biliary catheter.

In U.S. Pat. No., 5,029,580, titled, Medical aspirating apparatus with multi-lumen catheter tube and methods, invented by Radford, et. al., an aspirating/ventilating or aspirating apparatus comprising multi-lumen catheter tubes, for evacuation of lung secretions and for other functions as well. Two and three lumen catheter tubes which comprise three unique distal catheter end structures are disclosed. Material comprising oxygen-containing gas is introduced into the lungs under positive pressure through a first lumen of the catheter tube at a predetermined rate while secretions (and gas) are simultaneously aspirated from the lung into a second lumen by force of negative pressure. Oxygen gas inflow is controlled to equal gas outflow to maintain the partial pressure of oxygen into the lung at a high respiratory level. In a first distal catheter end structure, an oxygen-containing gas is introduced through the first lumen as a circumscribing jet around the second lumen to provide fluid flow turbulence external to a second lumen to improve homogenization of localized secretions resulting in more efficient aspiration. In a second distal catheter end structure, oxygen gas inflow is introduced into a common chamber at the distal end of the catheter, where effluent secretions are being aspirated, causing gas produced homogenization in the turbulence created by influent/effluent flow at the common distal catheter entry/exit port. In a third distal catheter end structure in a three lumen catheter, provision is made for independent introduction of medication and/or lavage solutions into the lungs via a third lumen into a common entry/exit port. Provision is made for monitoring pressures and flow rates through the catheter tube.

In U.S. Pat. No., 4,300,550, titled, Suction and oxygenation catheter, invented by Gandi, et. al., a catheter for use in simultaneous tracheal oxygenating and aspirating a patient. The catheter includes an elongated flexible tube arrangement adapted to have at least one end portion extended into the tracheal passage of a patient. The tube arrangement has two separate lumens there through. One lumen has at least one opening in the one end portion and is adapted to be connected at the other end of the tube arrangement to a source of suction to permit aspiration of material from the patient through the one lumen. The other lumen has at least one opening at the other end and is adapted to be connected at the other end to a source of oxygen to permit simultaneous oxygenating of the patient through the other lumen while the patient is aspirated through the one lumen.

The aforementioned patents differ from the present invention because the patented inventions lack the following features: Aspirate Sensor Device with stainless steel electrodes. An Activation Device electrically connected to the Aspirate Sensor Device. The Activation Device has three ports: a short tube port, suction trap port, and open port. The Activation Device connects to a standard suction trap via the suction trap port. Three chambers located within the Activation Device. The three chambers are short tube chamber, open tube chamber, and suction trap chamber. A valve door separates the open and short tube chambers. In the open position, the open chamber is continuous with the suction trap chamber.

Numerous innovations for an aspirator have been provided in the prior art that are adapted to be used. Even though these innovations may be suitable for the specific individual purposes to which they address, they would not be suitable for the purposes of the present invention as heretofore described.

### SUMMARY OF THE INVENTION

The present invention relates to an aspirator. More particularly, the present invention relates to an aspirator having a sensor.

The types of problems encountered in the prior art are apirators cannot detect a fluid level in a patient's esophagus lumen.

In the prior art, unsuccessful attempts to solve this problem were attempted namely: aspirators with stopping devices. However, the problem was solved by the present invention because it incorporates an activator sensor.

Innovations within the prior art are rapidly being exploited in the field of health care.

The present invention went contrary to the teaching of the art which describes and claims aspirators having timers and various other stopping devices.

The present invention solved a long felt need for an aspirator with a sensor.

Accordingly, it is an object of the present invention to provide an aspirator having a first tube, second tube and activator.

More particularly, it is an object of the present invention to provide a first tube having a first tube first end and a first tube second end with a first tube second end opening.

In keeping with these objects, and with others which will become apparent hereinafter, one feature of the present invention resides, briefly stated, in the second tube having a second tube first end and a second tube first end flush port with a second tube second end.

When the activator is designed in accordance with the present invention, it has an activator sensor, activator wire, activator battery housing, activator suction ingress, activator suction egress, activator inlet port, and activator switch.

In accordance with another feature of the present invention, the activator sensor has an activator sensor positive lead and an activator sensor negative lead.

Another feature of the present invention is that the activator switch has an activator switch pivot plate, activator switch lever with activator switch lever perforated cover, and activator switch alarm.

The novel features which are considered characteristic for the invention are set forth in the appended claims. The invention itself, however, both as to its construction and its method of operation, together with additional objects and advantages thereof, will be best understood from the following description of the specific embodiments when read and understood in connection with the accompanying drawings.

LIST OF REFERENCE NUMERALS
UTILIZED IN THE DRAWINGS

10-aspirator (10)
12-first tube (12)
12A-first tube first end (12A)
12B-first tube second end (12B)
12BA-first tube second end opening (12BA)
14-second tube (14)
14A-second tube first end (14A)
14AA-second tube first end flush port (14AA)
14B-second tube second end (14B)
16-activator (16)
16A-activator sensor (16A)
16AA-activator sensor positive lead (16AA)
16AB-activator sensor negative lead (16AB)
16B-activator wire (16B)
16C-activator battery housing (16C)
16D-activator suction ingress (16D)
16E-activator suction egress (16E)
16F-activator inlet port (16F)
16G-activator switch (16G)
16GA-activator switch pivot plate (16GA)
16GB-activator switch lever (16GB)
16GBA-activator switch lever perforated cover (16GBA)
16GH-activator switch alarm (16GH)
18-nose (18)
20-nasopharynx (20)
22-esophagus lumen (22)
24-stomach (24)
26-fluid (26)

### BRIEF DESCRIPTION OF THE DRAWINGS

FIG. 1 is a cross sectional view of a patient with an aspirator (10) inserted therein.

FIG. 2 is an enlarged view of a first tube second end (12B) displaying first tube second end openings (12BA).

FIG. 3 is an enlarged view of a second tube (14) displaying an activator sensor (16A) inserted therein.

FIG. 4 is a cross sectional view of a patient's esophagus lumen (22) displaying fluid (26) level therein to the height of the activator sensor (16A).

FIG. 5 is a cross sectional view of an aspirator (10) along line 5-5 of FIG. 1.

FIG. 6A is a cross sectional view of an activator (16) displaying the activator switch (16G) in a closed position.

FIG. 6B is a cross sectional view of an activator (16) displaying the activator switch (16G) in an open position.

### DESCRIPTION OF THE PREFERRED EMBODIMENT

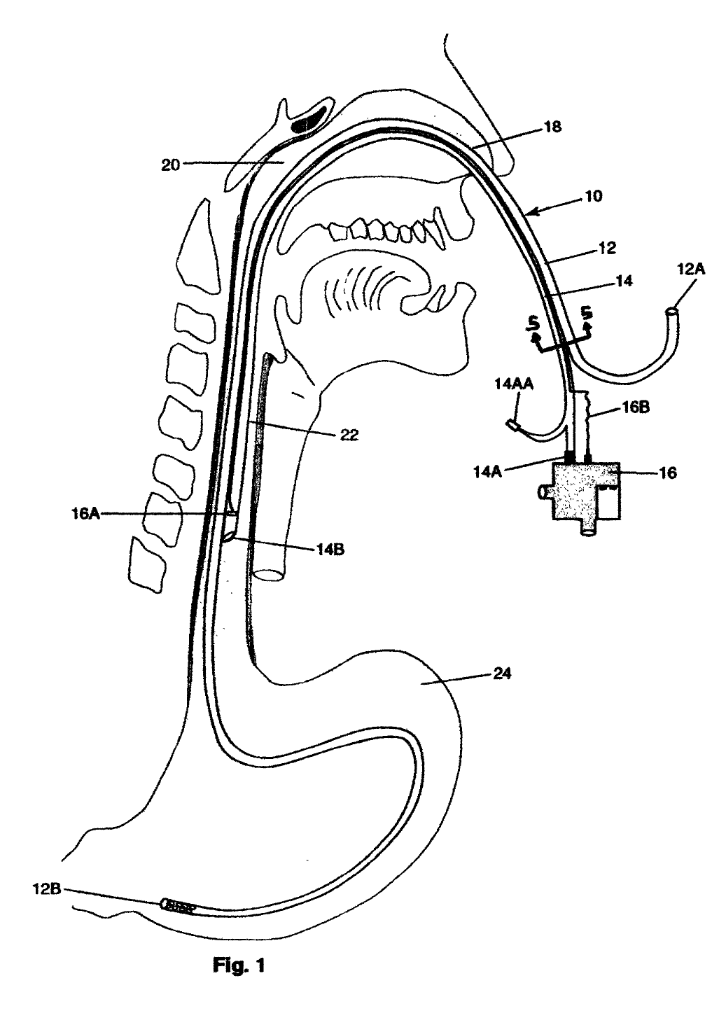

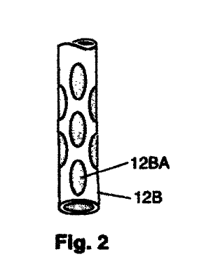

Firstly, referring to FIG. 1 which is a cross sectional view of a patient with an aspirator (10) inserted therein. The aspirator (10) comprises a first tube (12) insertable in a patient's nose (18) through a patient's nasopharynx (20) and esophagus lumen (22) into a patient's stomach (24). The first tube (12) comprises a first tube first end (12A) and a first tube second end (12B). The first tube second end (12B) preferably comprises a plurality of first tube second end openings (12BA) functioning to maintain at least one opening on the first tube second end (12B) if food or other debris is present in a patient's stomach (24) (refer to FIG. 2 which is an enlarged view of a first tube second end (12B) displaying first tube second end openings (12BA)).

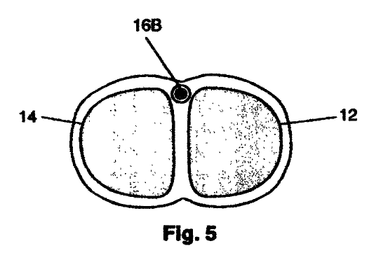

The aspirator (10) further comprises a second tube (14) insertable in the patient's nose (18) through the patient's nasopharynx (20). The second tube (14) comprises a second tube first end (14A) and a second tube second end (14B) positioned within the patient's esophagus lumen (22). The second tube first end (14A) preferably comprises a second tube first end flush port (14AA) functioning to provide a means to inject fluid into the second tube (14) to dislodge food or other debris from the second tube second end (14B). The first tube (12) and second tube (14) and activator wire (16B) are preferably incorporated into a compact integrated unit. (refer to FIG. 5 which is a cross sectional view of an aspirator (10) along line 5-5 of FIG. 1.)

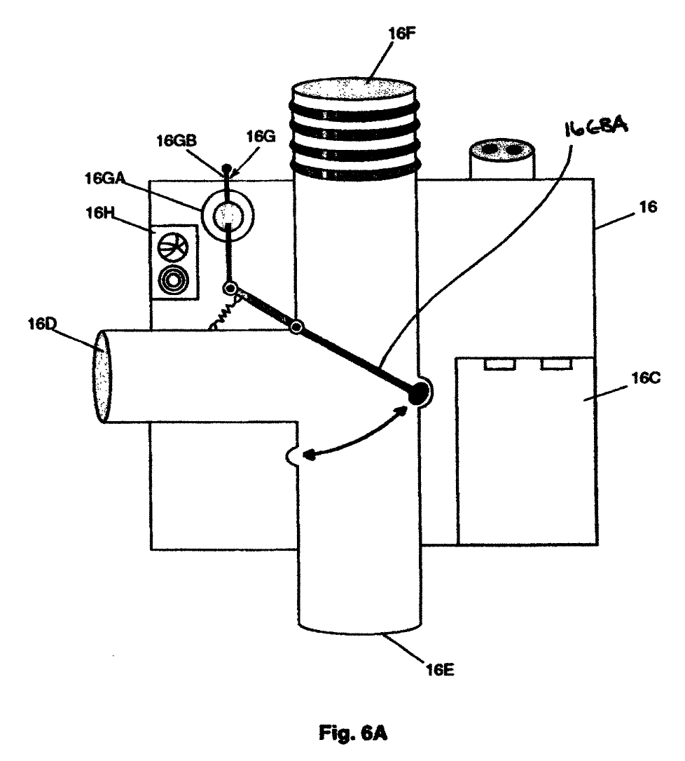

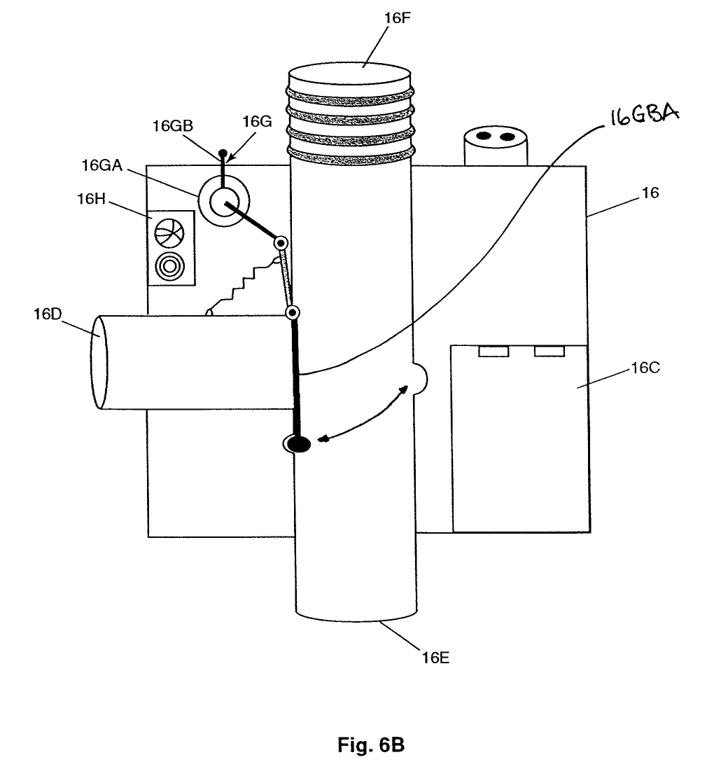

Now referring to FIGS. 6A and 6B which are cross sectional views of an activator (16) displaying the activator switch (16G) in a closed and open position, respectively. The aspirator (10) further comprises an activator (16) which comprises an activator sensor (16A) positioned within the second tube (14) at a distal end thereof.

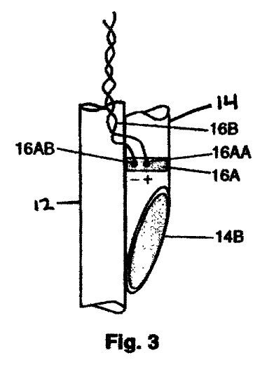

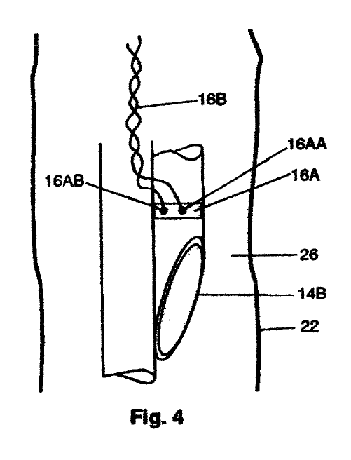

Referring to FIG. 3 which is an enlarged view of a second tube (14) displaying an activator sensor (16A) inserted therein and FIG. 4 which is a cross sectional view of a patient's esophagus lumen (22) displaying fluid (26) level therein to the height of the activator sensor (16A), the activator sensor (16A) comprises an activator sensor first lead (16AA) and an activator sensor second lead (16AB) electrically connected to a proximal end of an activator wire (16B) which is electrically connected at a distal end to a vacuum pump (not shown). The activator (16) further comprises an activator suction ingress (16D). The activator (16) further comprises an activator suction egress (16E) sealably connected to a collection container (not shown) which is sealably connected to the vacuum pump (not shown). The activator (16) further comprises an activator inlet port (16F) sealably connected to the second tube first end (14A). The activator (16) further comprises an activator switch (16G) which comprises a activator switch pivot plate (16GA) movably connected to an activator switch lever (16GB) having an activator switch lever perforated cover (16GBA) positioned within and having a complimentary configuration to the activator suction egress (16E). When the activator switch (16G) is in a closed position, the activator switch lever perforated cover (16GBA) seals the activator suction egress (16E) preventing suction from the second tube (14). When the activator switch (16G) is in an open position, the activator switch lever perforated cover (16GBA) seals the activator suction ingress (16D) allowing suction from the second tube (14) into the collection container (not shown). The activator (16) comprises an activator switch alarm (16GH) electrically connected to a power means and the activator wire (16B) functioning to alert a care giver when a fluid (26) level in a patient's esophagus lumen (22) reaches the activator sensor (16A). The activator (16) further comprises a portable power means contained within an activator battery housing (16C).

It will be understood that each of the elements described above, or two or more together, may also find a useful application in other types of constructions differing from the type described above.

While the invention has been illustrated and described as embodied in an aspirator, it is not intended to be limited to the details shown, since it will be understood that various omissions, modifications, substitutions and changes in the forms and details of the device illustrated and in its operation can be made by those skilled in the art without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

What is claimed as new and desired to be protected by Letters Patent is set forth in the appended claims.

What is claimed is:

1. An aspirator (10) comprising:
A) a first tube (12) insertable in a patient's nose (18) through a patient's nasopharynx (20) and esophagus lumen (22) into a patient's stomach (24), the first tube (12) comprises a first tube first end (12A) and a first tube second end (12B);
B) a second tube (14) insertable in the patient's nose (18) through the patient's nasopharynx (20), the second tube (14) comprises a second tube first end (14A) and a second tube second end (14B) positioned within the patient's esophagus lumen (22); and
C) an activator (16) which comprises an activator sensor (16A) positioned within the second tube (14) at a distal end thereof, the activator sensor (16A) comprises an activator sensor first lead (16AA) and an activator sensor second lead (16AB) electrically connected to a proximal end of an activator wire (16B) which is electrically connected at a distal end to a vacuum pump (not shown), the activator (16) further comprises an activator suction ingress (16D), the activator (16) further comprises an activator suction egress (16E) sealably connected to a collection container (not shown) which is sealably connected to the vacuum pump (not shown), the activator (16) further comprises an activator inlet port (16F) sealably connected to the second tube first end (14A), the activator (16) further comprises an activator switch (16G) which comprises a activator switch pivot plate (16GA) movably connected to an activator switch lever (16GB) having an activator switch lever perforated cover (16GBA) positioned within and having a complimentary configuration to the activator suction egress (16E), when the activator switch (16G) is in a closed position, the activator switch lever perforated cover (16GBA) seals the activator suction egress (16E) preventing suction from the second tube (14), when the activator switch (16G) is in an open position, the activator switch lever perforated cover (16GBA) seals the activator suction ingress (16D) allowing suction from the second tube (14) into the collection container (not shown).

2. The aspirator (10) as described in claim 1, wherein the first tube second end (12B) comprises a plurality of first tube second end openings (12BA) functioning to maintain at least one opening on the first tube second end (12B) if food or other debris is present in a patient's stomach (24).

3. The aspirator (10) as described in claim 1, wherein the second tube first end (14A) comprises a second tube first end flush port (14AA) functioning to provide a means to inject fluid into the second tube (14) to dislodge food or other debris from the second tube second end (14B).

4. The aspirator (10) as described in claim 1, wherein the activator (16) comprises an activator switch alarm (16GH) electrically connected to a power means and the activator wire (16B) functioning to alert a care giver when a fluid (26) level in a patient's esophagus lumen (22) reaches the activator sensor (16A).

5. The aspirator (10) as described in claim 4, wherein the activator (16) further comprises a portable power means contained within anactivator battery housing (16C).
